SEQUENCE LISTING (1 sequence)

SEQ ID NO: 1 - DNA, 1398 bp, Artificial Sequence
  anti-CD19-41BB
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccaccactcccgcaccccgccctcctactcctgcccctaccattgctagccaaccgcttagtctgagacctgaggcctgtaggcccgctgctggtggcgctgtgcacacccgaggattggacttcgcttgcgacatctacatctgggcacctctggctgggacctgcggcgtgttgttgttgagcctggtgattacgctgtactgtaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgc

(19) United States
(12) Patent Application Publication
(10) Pub. No.: US 2022/0401465 A1
(43) Pub. Date: Dec. 22, 2022

(54) USES OF NAD+ AND/OR NAD+ INHIBITORS AND/OR NAD+ AGONISTS AND COMBINATION PREPARATION THEREOF

(71) Applicant: ShanghaiTech University, Shanghai (CN)

(72) Inventors: Gaofeng FAN, Shanghai (CN); Haopeng WANG, Shanghai (CN); Yuetong WANG, Shanghai (CN); Fei WANG, Shanghai (CN)

(73) Assignee: ShanghaiTech University, Shanghai (CN)

(21) Appl. No.: 17/616,200

(22) PCT Filed: Jun. 4, 2019
(86) PCT No.: PCT/CN2019/090022

§ 371 Date: Aug. 19, 2022

(51) Int. Cl. A61K 31/7084; A61K 38/17; A61K 35/17; A61K 45/06

(52) U.S. Cl. CPC A61K 31/7084; A61K 38/1774; A61K 35/17; A61K 45/06

(57) ABSTRACT

Disclosed are uses of NAD+ and/or NAD+ agonists and/or NAD+ inhibitors for preparing a preparation or a kit, and a combination preparation including T cells and NAD+ and/or NAD+ agonists and/or NAD+ inhibitors. The preparation or kit is used for regulating T cell activity, regulating the expression level of CD69 on the surface of T cells, and/or regulating the phosphorylation level in T cells, and/or treating diseases related to T cell activity.

### TECHNICAL FIELD

The present disclosure relates to the field of biomedicine, in particular to an use of NAD+ and/or NAD+ inhibitors and/or NAD+ agonists in regulating T cell activity.

### BACKGROUND

Cancer is a major public health problem worldwide and has gradually replaced cardiovascular disease as the leading cause of death. It's urgent to develop therapeutic strategies for cancer treatment. In recent years, immunotherapy has achieved revolutionary results in the clinical treatment of cancer. At present, clinical immunotherapy mainly includes immune checkpoint blockade and adoptive T cell therapy. The strategy of using immune checkpoint blockade has achieved impressive results in the clinical treatment of cancer such as melanoma, non-small cell lung cancer, and head and neck squamous cell carcinoma. However, only about 20%-40% of patients initially respond to these inhibitors, and a significant proportion of initial responders will eventually relapse several months or years later. Chimeric Antigen Receptor T Cell (CAR-T) therapy is to obtain T cells from the patient, carry out genetic modification, and then transfer the modified T cells into the patient, activating an anti-tumor immune response. Based on the differences in CAR modification, five major types of CAR-T cells have been approved by the FDA or undergoing clinical trials. Upon activation, CAR-T cells can differentiate into memory cells with a longer life span, which makes this therapeutic strategy remarkable success in the treatment of hematological malignancies. But the application of this treatment method is limited in solid tumors, and patients cannot achieve durable benefits.

The research progress in the field of the tumor microenvironment and tumor immunology has revealed the mechanism of immune escape and the complex regulatory network of the immune response, including: 1. Tumor cells can escape immune surveillance through the high expression of PD-L1 and other immune checkpoint ligands. Based on these findings, PD-1 and CTLA-4 antibodies have emerged and produced curative effects in cancer patients. In addition, newly identified immune checkpoints are also undergoing clinical trials (such as LAG-3, TIM-3, and VISTA); 2. Mesenchymal cells, immunosuppressive monocytes, macrophages, etc., in the tumor microenvironment can inhibit T cell infiltration by recruiting T cells in the stroma or wrapping the tumor cells and regulating T cell differentiation. Based on this, cytokine-specific inhibitors combined with immune checkpoint blockade are often used in the clinic; 3. It is noteworthy that the tumor microenvironment can also regulate the activity of immune cells by metabolic competition. For example, tumor cells prefer anaerobic glycolysis, which significantly increases the concentration of lactic acid in the tumor microenvironment and induces tumor-associated macrophages differentiated into M2 types, thereby inhibiting T cell activity. In the past decades, researchers have shown that during the immune response process, the proliferation and differentiation of T cells are closely related to metabolic regulation. When T cells recognize antigens, the immune response is initiated, and the cells change from a relatively quiescent state to a highly active state. With the decrease of the antigen load, most activated T cells initiate the death program, while a small number of long-life memory T cells persist over time and remain in a relatively quiescent state. The metabolic activity in T cells also changes with the states of the T cells. For example, when T cells are in a relatively quiescent state (such as naïve T cells or memory T cells), the cells mainly rely on catabolism, so that nutrients are completely degraded to generate the required energy, such as pyruvate metabolism (TCA). While in activated T cells, in order to address the need for more energy and cytokine synthesis, the cells rely on glycolysis to produce energy. Therefore, in the process of T cell activation, T cells undergo a transformation from TCA metabolism dependent on mitochondrial activity to anaerobic glycolysis. For example, upon anti-CD28 stimulation, T cells promote the expression of carnitine palmitoyltransferase 1A (CPT1A), enhance oxidation of mitochondrial fatty acid, elongate mitochondrion, and decrease mitochondrial crista spacing. During the recovery of T cells to the resting state, mitochondria gradually become shorter, and the internal crista structure becomes loose. However, the regulation of the tumor microenvironment on the metabolic process in immune cells during anti-tumor immune response still needs further studies. Improving the metabolism of T cells to enhance the tumor-killing ability of T cells has become a focus of current research.

The important role of nicotinamide adenine dinucleotide (NAD+) in delaying aging has attracted widespread attention. Nowadays, studies have shown that NAD+ is correlated with tumor cells and cancer, and it has become an important research topic in this field.

### SUMMARY

The present invention provides uses of NAD+ and/or NAD+ inhibitors and/or NAD+ agonists in regulating T cell activity.

In an aspect, the present invention provides the use of NAD+ and/or NAD+ agonists and/or NAD+ inhibitors in the preparation of preparation or kit. The preparation or kit is used for:
(1) regulating the activity of T cells; and/or,
(2) regulating the expression level of CD69 on the surface of T cells; and/or,
(3) regulating the phosphorylation level in T cells; and/or,
(4) treating the disease related to T cell activity.

In some embodiments of the present invention, the NAD+ agonist is one or more of NAD+ precursor agonists, nicotinamide phosphate ribosyltransferase agonists, PARP inhibitors, SIRT inhibitors, CD38 inhibitors, and NAD+ metabolic enzyme inhibitors.

In some embodiments of the present invention, the NAD+ inhibitor is one or more of nicotinamide phosphate ribosyltransferase inhibitors, NAD synthase 1 inhibitors, and SIRT agonists.

In some embodiments of the present invention, the preparation or kit is used to regulate NAD+ level or NAD+ activity in T cells.

In some embodiments of the present invention, the regulation includes positive regulation and negative regulation.

In some embodiments of the present invention, the T cell activity is specifically the cytotoxic ability of T cells, and the cytotoxic ability is the tumor cell-killing ability.

In some embodiments of the present invention, the T cell is referred to as CAR-T cell and TCR-T cell.

In some embodiments of the present invention, the disease related to T cell activity is selected from disease related to excessively low T cell activity or disease related to excessively high T cell activity.

In some embodiments of the present invention, the disease related to T cell activity is selected from the group consisting of T cell inhibitory inflammation, low immune response, tumor, infectious disease, autoimmune disease, T cell-mediated inflammation, and transplant rejection.

Another aspect of the present invention provides a regulation method, which is used for:
(1) regulating the activity of T cells; and/or,
(2) regulating the expression level of CD69 on the surface of T cells; and/or,
(3) regulating the phosphorylation level in T cells; and/or,
the regulation method specifically includes: regulating the intracellular level or activity of NAD+ to regulate the activity of T cells.

In some embodiments of the present invention, the method specifically includes: subjecting T cells to the presence of NAD+, NAD+ inhibitors, and/or NAD+ agonists, and the NAD+ inhibitor is one or more of nicotinamide phosphate ribosyltransferase inhibitors, NAD synthase 1 inhibitors, and SIRT agonists, the NAD+ agonist is one or more of NAD+, NAD+ precursor agonists, nicotinamide phosphate ribosyltransferase agonists, PARP inhibitors, SIRT inhibitors, CD38 inhibitors, and NAD+ metabolic enzyme inhibitors;

In some embodiments of the present invention, the regulation is in vitro regulation.

In some embodiments of the present invention, the regulation includes positive regulation and negative regulation.

In some embodiments of the present invention, the T cell activity is the cytotoxic ability of T cells, preferably tumor cell-killing ability.

In some embodiments of the present invention, the T cell includes CAR-T cell and TCR-T cell.

Another aspect of the present invention provides a combination preparation. The combination preparation includes: T cells, and/or NAD+ and/or NAD+ agonists and/or NAD+ inhibitors.

In some embodiments of the present invention, the NAD+ inhibitor is one or more of nicotinamide phosphate ribosyltransferase inhibitors, NAD synthase 1 inhibitors, and SIRT agonists

In some embodiments of the present invention, the NAD+ agonist is one or more of NAD+, NAD+ precursor agonists, nicotinamide phosphate ribosyltransferase agonists, PARP inhibitors, SIRT inhibitors, CD38 inhibitors, and NAD+ metabolic enzyme inhibitors

In some embodiments of the present invention, the T cell includes CAR-T cell and TCR-T cell.

Another aspect of the present invention provides the use of combination preparation in the preparation of medicines.

In some embodiments of the present invention, the medicine is selected from medicines used to treat diseases related to T cell activity.

### BRIEF DESCRIPTION OF THE DRAWINGS

FIG. 1 shows a schematic diagram of the activation ability of T cells metabolism-regulated by NAD+ in Example 1 of the present invention.

FIG. 2 shows a schematic diagram of the in vitro cytotoxic ability of T cells metabolism-regulated by NAD+ in Example 2 of the present invention.

FIG. 3 shows a schematic diagram of the combination of NAD+ metabolic precursor Nicotinamide (NAM) combined with CAR-T to enhance tumor treatment effect in Example 3 of the present invention.

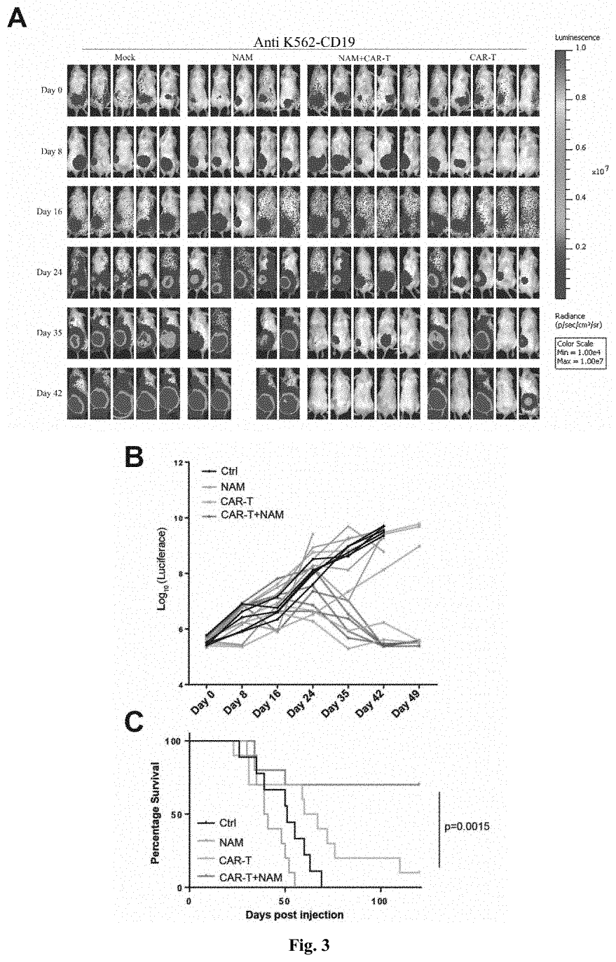

### DETAILED DESCRIPTION

Through a large number of experimental studies, the inventors unexpectedly discovered that the substance used to regulate NAD+ levels could affect the expression level of CD69 on the surface of T cells and the phosphorylation level of T cells, thereby significantly affecting the activity of T cells. On this basis, the present invention was completed.

The first aspect of the present invention provides the use of NAD+ and/or NAD+ agonists and/or NAD+ inhibitors in the preparation of preparation or kit. The preparation or kit is used for: regulating the activity of the T cells; and/or, regulating the expression level of CD69 on the surface of T cells; and/or, regulating the phosphorylation level in T cells; and/or, treating diseases related to T cell activity. NAD+ (Nicotinamide adenine dinucleotide, coenzyme I) is a nucleotide coenzyme. The regulation of T cell activity can be reflected by the (content) level of NAD+ in T cells, and the regulation may be positive regulation. For example, NAD+ and/or NAD+ agonists can be used to increase NAD+ intracellular levels and/or increase NAD+ activity, thereby increasing T cell activity, up-regulating the expression level of CD69 on the surface of T cells, or up-regulating the phosphorylation level in T cells; the regulation may also be negative regulation, for example, NAD+ inhibitors can be used to reduce NAD+ intracellular levels and/or reduce NAD+ activity, thereby reducing T cell activity, down-regulating the expression level of CD69 on the surface of T cells, or down-regulating the phosphorylation level in T cells.

In the present invention, the NAD+ inhibitor generally refers to a substance that can reduce the intracellular (content) level of NAD+ and/or reduce the activity of NAD+. The type of NAD+ inhibitor should be known to the person in this field, for example, the NAD+ inhibitor may include but is not limited to one or more of nicotinamide phosphate ribosyltransferase inhibitors, NAD synthase 1 inhibitors, and SIRT agonists. For another example, the nicotinamide phosphate ribosyltransferase inhibitor may specifically include but is not limited to STF-118804, GMX1778, KPT-9274, FK866, Nampt-IN-1, GNE-617 hydrochloride, GNE-617, CB30865, KPT-9247, etc.; for another example, the NAD synthase 1 inhibitor may specifically include but is not limited to NADSYN1i, etc.; for another example, the SIRT agonist may specifically include but is not limited to SRT 1720, CAY10602, MDL-801, Quercetin, SRT 2104, etc.

In the present invention, the substance capable of increasing the intracellular level of NAD+ and/or increasing the activity of NAD+ may be an NAD+ agonist and/or NAD+. The types of NAD+ agonists should be known to the person in this field. For example, in addition to NAD+, the NAD+ agonists include but are not limited to one or more of NAD+ precursor agonists, nicotinamide phosphate ribosyltransferase agonists, PARP inhibitors, SIRT inhibitors, CD38 inhibitors, NAD+ metabolic enzyme inhibitors, etc.; for another example, the NAD+ precursor agonist may include but is not limited to one or more of Nicotinamide (NAM), nicotinic acid (NA), nicotinic acid mononucleotide (NAMN), tryptophan (TRP), Nicotinamide mononucleotide (NMN), quinolinic acid (QA), nicotinamide riboside (NR), etc.; for another example, the nicotinamide phosphate ribosyltransferase agonist may specifically be P7C3, etc.; for another example, the PARP inhibitor may specifically include but is not limited to PARP-2-IN-1, 3-Aminobenzamide, UPF 1069, Veliparib, AZD-2461, E7449, Rucaparib, Olaparib, Talazoparib tosylate, A-966492, AG14361, NMS-P118, Pamiparib, Iniparib, etc.; for another example, the SIRT inhibitor may specifically include but is not limited to SIRT-IN-2, AGK2, Tenovin 6 Hydrochloride, OSS_128167, 3-TYP, Salermide, AK-7, etc.; for another example, the CD38 inhibitor may specifically include but is not limited to CD38 inhibitor 1, Apigenin, etc.; for another example, the NAD+ metabolic enzyme inhibitor may specifically include but is not limited to ACMSD inhibitor, etc., and the ACMSD inhibitor may specifically include, but is not limited to TES-1025, TES-991, etc.

In the present invention, the regulation of T cell activity can be reflected by the expression level of CD69 on the surface of T cells. The regulation includes positive regulation and negative regulation, for example, NAD+ inhibitors can reduce the expression level of CD69 on the surface of T cells, and a decrease in the expression level of CD69 on the cell surface generally means a decrease in the activity of T cells; for another example, NAD+ agonists can increase the expression level of CD69 on the surface of T cells, and an increase in the expression level of CD69 on the surface of T cells generally means an increase in the activity of T cells.

In the present invention, the regulation of T cell activity can be reflected by the level of (tyrosine) phosphorylation in T cells. The regulation includes positive regulation and negative regulation. For example, NAD+ inhibitors can reduce the phosphorylation level in T cells, and a decrease in the level of phosphorylation in T cells generally means a decrease in the activity of T cells; for another example, NAD+ agonists can increase the phosphorylation level in T cells, and an increase in the level of phosphorylation in T cells generally means an increase in the activity of T cells.

In the present invention, the T cell generally refers to a CD3+ T cell, and the activity of the T cell generally refers to the cytotoxic ability of the T cell, preferably the cytotoxic ability of the target cells, and the target cells may generally be tumor cells. The T cells may also be obtained through gene transfer technology, including but not limited to CAR-T cell (Chimeric Antigen Receptor T-Cell), TCR-T cell (T cell receptor chimeric T cell), etc. The CAR-T cell is generally the T cell with modified receptors on the surface of the cell membrane. The membrane-bound receptor generally includes an extracellular domain, and may also include an extracellular hinge region, a transmembrane region, and an intracellular signal region. The extracellular domain may generally include molecules targeting target cells (tumor-associated antigen binding regions). The TCR-T cell generally refers to a T cell transduced by a T cell receptor, and it can recognize an antigen inside or on the surface of a target cell through the T cell receptor, thereby targeting the target cell. In another embodiment of the present invention, the T cell is a CAR-T cell, and the extracellular domain of the CAR-T cell includes an anti-CD19 single-chain variable fragment (scFv); thus, it can target the tumor cell expressing CD19, for example, the tumor cell expressing CD19 may specifically be CD19-positive B cell malignancies, B-cell chronic lymphocytic leukemia (CLL), and B-cell non-Hodgkin's lymphoma (NHL), etc. By regulating the activity of T cells, the activity of CAR-T cells can be further regulated. In an embodiment of the present invention, the activity-regulated CAR-T cells have stronger tumor cell-killing ability, significantly inhibit the growth of tumor cells, and increase the survival time of tumor-bearing mice.

In the preparation or kit provided by the present invention, the NAD+ and/or NAD+ inhibitor and/or NAD+ agonist may be used as a single active ingredient, or may be combined with other active components (i.e., other components except for NAD+, NAD+ inhibitors and NAD+ agonists) to participate in the regulation of the activity of T cells, the expression level of CD69 on the surface of T cells, and the phosphorylation level in T cells, or participate in the treatment of diseases related to T cell activity.

In the present invention, the preparation or kit may be used for the treatment of diseases related to T cell activity, and the term “treatment” includes preventive, curative or palliative treatments that can lead to desired pharmaceutical and/or physiological effects. The effect preferably refers to medically reducing one or more symptoms of the disease, or completely eliminating the disease, or blocking or delaying the occurrence of the disease, and/or reducing the risk of disease development or deterioration. The disease related to T cell activity may specifically be a disease related to excessively high T cell activity and/or a disease related to insufficient T cell activity. The disease related to insufficient T cell activity may be inhibited inflammation, low immune response, tumor, or infectious disease. The disease related to excessively high T cell activity may be an autoimmune disease, T cell-mediated inflammation, transplant rejection, etc. The tumor may specifically include but is not limited to blood cancer, bone cancer, lymphoma (including lymphocyte carcinoma), intestinal cancer, liver cancer, gastric cancer, pelvic cancer (including uterine cancer, cervical cancer), lung cancer (including mediastinal cancer), brain cancer, nerve cancer, breast cancer, esophageal cancer, kidney cancer, etc.

The second aspect of the present invention provides a regulation method, which can be used to regulate the activity of T cells. The regulation of the activity of T cells can be reflected by the expression level of CD69 on the surface of T cells, and the regulation of the activity of T cells can also be reflected by the up-regulating the phosphorylation level in T cells. The regulation method includes: regulating the intracellular level or activity of NAD+ to regulate the activity of T cells and/or the expression level of CD69 on the surface of T cells and/or the level of phosphorylation in the T cells. In the regulation method, the T cell activity may specifically be the cytotoxic ability of the T cell, etc., which can be reflected by the expression level of CD69 on the surface of the T cells and/or the phosphorylation level in the T cells, and the T cell may also include CAR-T cell and TCR-T cell. The regulation of T cell activity includes positive regulation and negative regulation. For example, it may be increasing T cell activity and/or decreasing T cell activity.

For the person in this field, a suitable method may be selected to regulate the intracellular NAD+ level or NAD+ activity of T cells. These methods may be in vitro regulation methods. For example, T cells may be placed in the presence of exogenous NAD+, and/or NAD+ inhibitors and/or NAD+ agonists. In a preferred embodiment of the present invention, the usage amount of the NAD+ and/or NAD+ agonists may be 50˜150 μM, and the usage amount of NAD+ inhibitors may be 10˜1000 nM. In another preferred embodiment of the present invention, exogenous NAD+, and/or NAD+ inhibitors, and/or NAD+ agonists may be added directly to the medium. These methods may also be in vivo methods. For example, exogenous NAD+, and/or NAD+ inhibitors, and/or NAD+ agonists may be administered to the individual. These methods may also be in vivo regulation methods. For example, they may be in vivo regulation at the level of a mouse model.

In the regulation method provided by the present invention, the NAD+ inhibitor may be various NAD+ inhibitors as described in the first aspect of the present invention, and the NAD+ agonist may be various NAD+ agonists as described in the first aspect of the present invention. The exogenous NAD+, and/or NAD+ inhibitors and/or NAD+ agonists may be used as a single active ingredient to regulate T cell activity, or may be combined with other components which can be used to regulate T cell activity to participate in the regulation of T cell activity.

The third aspect of the present invention provides a composition, which includes NAD+, and/or NAD+ inhibitors and/or NAD+ agonists, and the composition may be used for: regulating the activity of the T cells; and/or, regulating the expression level of CD69 on the surface of T cells; and/or, regulating the phosphorylation level in T cells; and/or, treating the disease related to T cell activity. In the composition, the NAD+, and/or NAD+ inhibitors and/or NAD+ agonists and mechanism thereof may refer to the relevant content in the first aspect of the present invention. In the pharmaceutical composition, NAD+, and/or NAD+ inhibitors and/or NAD+ agonists may be used as a single active ingredient, or may be combined with other active ingredients.

The fourth aspect of the present invention provides a combination preparation. The combination preparation includes: T cells, and NAD+ and/or NAD+ agonists, and/or NAD+ inhibitors. The T cell may be a CAR-T cell, the NAD+ inhibitors may be various NAD+ inhibitors as described in the first aspect of the present invention, and the NAD+ agonists may be various NAD+ agonists as described in the first aspect of the present invention. Administrating NAD+ inhibitors and/or NAD+ agonists to the individual and administrating the activated CAR-T cells to the individual at the same time can regulate the activity of CAR-T cells, so that the CAR-T cells have stronger tumor cell-killing ability, and can significantly inhibit the growth of tumor cells, and increase the survival time of tumor-bearing mice.

The fifth aspect of the present invention provides the use of the combination preparation provided in the fourth aspect of the present invention in the preparation of medicines. The medicine may generally be used to treat diseases related to T cell activity. The disease related to T cell activity may specifically be a disease related to excessively high T cell activity and/or a disease related to insufficient T cell activity. The disease related to insufficient T cell activity may be inhibited inflammation, low immune response, tumor, or infectious disease. The disease related to excessively high T cell activity may be an autoimmune disease, T cell-mediated inflammation, transplant rejection, etc. The tumor may specifically include, but is not limited to blood cancer, bone cancer, lymphoma (including lymphocyte carcinoma), intestinal cancer, liver cancer, gastric cancer, pelvic cancer (including uterine cancer, cervical cancer), lung cancer (including mediastinal cancer), brain cancer, nerve cancer, breast cancer, esophageal cancer, kidney cancer, etc.

The sixth aspect of the present invention provides a treatment method, which includes: administering a therapeutically effective amount of NAD+, NAD+ inhibitors, NAD+ agonists, or the combination preparation provided in the fourth aspect of the present invention to an individual. The treatment method provided by the present invention may be used to treat indications including but not limited to tumors, autoimmune diseases, inflammatory reactions, infectious diseases, transplant rejection, etc. The tumor may specifically include, but is not limited to blood cancer, bone cancer, lymphoma (including lymphocyte carcinoma), intestinal cancer, liver cancer, gastric cancer, pelvic cancer (including uterine cancer, cervical cancer), lung cancer (including mediastinal cancer), brain cancer, nerve cancer, breast cancer, esophageal cancer, kidney cancer, etc.

In the present invention, “individual” generally includes human and non-human primates, such as mammals, dogs, cats, horses, sheep, pigs, cows, etc., which may benefit from the treating of the preparation, kit, or combination preparation.

In the present invention, “therapeutically effective amount” generally refers to an amount that can achieve the effect of treating the diseases listed above after a proper administration period.

The routes of administration of T cells, NAD+, NAD+ inhibitors, and NAD+ agonists should be known to the person in this field. For example, the NAD+, NAD+ inhibitors, or NAD+ agonists may be administered by oral, rectal, parenteral (intravenous, intramuscular, or subcutaneous, etc.), topical administration, etc . . . For another example, the T cell may be administered by intravenous injection. The dosage of the T cell, NAD+, NAD+ inhibitors, and NAD+ agonists is usually a safe and effective amount. For example, the dosage of the administration of NAD+ and/or NAD+ agonists may be 400-600 mg/kg/day; the dosage of the administration of NAD+ inhibitors may be 50-150 mg/kg/day; the dosage of the administration of T cell may be 0.5*10⁶−5*10⁶ cells/20 g.

The inventor of the present invention innovatively discovered that the activity of T cells could be regulated through the NAD+ metabolic pathway. In vitro experiments have proved that increasing the level of NAD+ can significantly improve the cytotoxic ability of T cells against tumor cells. In vivo experiments have further proved that supplementation of NAD+-related synthetic precursors can enhance the cytotoxic effect of T cells against tumors, thus proving that the combination of NAD+ and chimeric antigen receptor T cells can significantly improve the effect of tumor immunotherapy, which is expected to solve the current ineffectiveness of chimeric antigen receptor T cell therapy in the treatment of solid tumors and has a promising industrialization prospect.

Embodiments of the present disclosure will be described below with specific examples, and other advantages and effects of the present disclosure may be easily understood by the person in this field from the disclosure in the specification. The present invention may also be carried out or applied in other different specific embodiments, and various modifications or changes may also be made to the details in the specification based on different ideas and applications without departing from the spirit of the present disclosure.

Before further describing the specific examples of the present invention, it should be understood that the scope of protection of the present invention is not limited to the following specific examples; it should also be understood that the terms used in the examples of the present invention are used to describe the specific examples, it is not intended to limit the scope of protection of the present invention; in the description and claims of the present invention, unless the context clearly indicates otherwise, the singular forms “a”, “one” and “this” include plural forms.

When numerical ranges appear in the examples, it should be understood that, unless otherwise specified in the present invention, the two endpoints of each numerical range and any value between the two endpoints may be selected. Unless otherwise defined, all technical and scientific terms used in the present invention have the same meaning as commonly understood by the person in this field. In addition to the specific methods, equipment, and materials used in the examples, any methods, equipment, and materials in this field that are similar or equivalent to the methods, equipment, and materials described in the examples of the present invention may also be used to realize the present invention according to mastery of the person in this field and the description of the present invention.

Unless otherwise specified, the experimental methods, detection methods, and preparation methods disclosed in the present invention all adopt conventional molecular biology, biochemistry, chromatin structure and analysis, analytical chemistry, cell culture, and recombinant DNA technology in the technical field and the conventional technology in the related field. These technologies have been well explained in the existing literature. For details, see Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol.304, Chromatin (PMWassarman and AP Wolfffe, eds.), Academic Press, San Diego, 1999; and METHODS IN MOLECULAR BIOLOGY, Vol. 119, Chromatin Protocols (PBBecker, ed.) Humana Press, Totowa, 1999 and so on.

### Embodiment 1

Regulation of T cell activation by NAD+ levels:

Human peripheral blood mononuclear cells (PBMC) were diluted 1:1 with fresh blood and normal saline (Meilun Biological MA0083) and then added flat to the upper layer of an equal volume of Histopaque®-1077 (Sigma10771) separation solution. The cells were centrifuged slowly for 30 min at 500 g at room temperature. After centrifugation, the white membrane layer between the plasma and the separation solution was imbibed and washed with normal saline. After repeated washing, the cells were resuspended with 10% FBS (Thermofisher 10099141C) RPMI (Corning10-040-CV) medium for later use.

The concentration of PBMC cells was adjusted to no more than 0.5 million/ml, and the final concentration of 1 μM FK866 (Selleck S2799), 100 μM NAD+ (Selleck S2518), or 1μM FK866, and 100 μM NAD+ were added respectively. The cells after drug addition were cultured in a cell incubator for 24 hours at 37° C., with 5% CO₂.

Highly adsorbed 96-well plates were coated with CD28 (Biolegend 102112), and CD3 (Thermofisher 14-0037-82) antibodies at the final concentration of 0 or 3 μg/ml, the PBMC cells treated with the above different treatments were added to the well plates, respectively. After 24 h stimulation, the cells were collected for CD69 staining. CD69 staining: after the cells were collected, the medium was centrifuged and removed, and the cells were stained on ice for 40 min with an anti-CD69-APC(Biolegend 310910) antibody, which was diluted at 1:800 with Staining buffer (Biolegend 420201), the staining solution was centrifuged and removed. After washing with Staining buffer, the cells were resuspended with staining buffer containing DAPI and then detected on BD LSRFortessa.

Highly adsorbed 96-well plates were coated with CD28 (Biolegend 102112), and CD3 (Thermofisher 14-0037-82) antibodies at the final concentration of 0 or 3 μg/ml, the PBMC cells treated with the above different treatments were added to the well plates, respectively. After 5 min stimulation (at the same time, phosphatase inhibitor sodium vanadate was added), the cells were collected for WB testing. WB testing: after cell collection, the cells were lysed to extract proteins, which were then electrophoretically transferred and detected with 4G10 antibody (Millipore16-103) for anti-Phosphotyrosine testing.

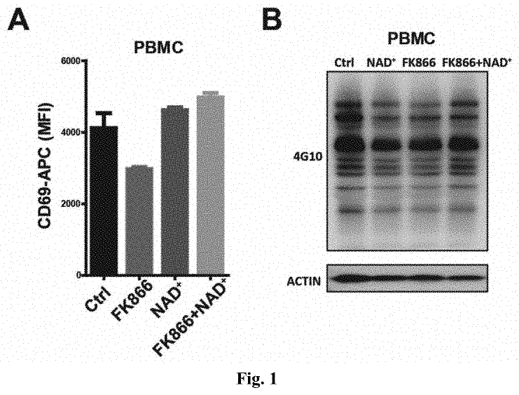

As mentioned above, human peripheral blood lymphocytes (PBMC) were used for the experiment. When T cells in PBMC were activated with anti-CD3, NAD+ synthesis inhibitor FK866 or solvent were administrated, respectively. Compared with the changes in NAD+ levels, since the expression of CD69 on the surface of the cell membrane will be enhanced and the intracellular overall phosphorylation level will be transiently enhanced after T cell activation, the effect of NAD+ level on the activation ability of human T cells can be verified by the change of expression of CD69 on the membrane surface of human T cells and intracellular phosphorylation level. The specific experimental results are shown in FIG. 1, which shows that: (a) the expression level of CD69 on the cell surface under different drug treatments after the PBMC is activated with anti-CD3 antibody; (b) the intracellular tyrosine phosphorylation level under different drug treatments after the PBMC is activated with anti-CD3 antibody. It was found that when NAD+ synthesis was inhibited, both CD69 on the surface of T cells and intracellular phosphorylation levels were significantly reduced, and T cell activation ability was significantly decreased.

### Embodiment 2

Regulation of the killing ability of T cells against tumor cells by NAD+ levels:

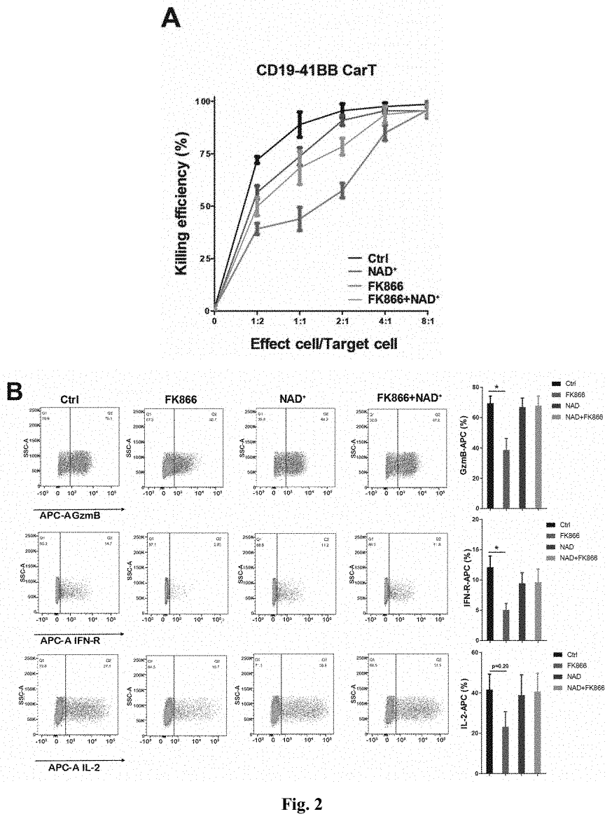

Construct an experimental model for verifying the cytotoxic ability of T cells in vitro to verify the effect of NAD+ level on the tumor-killing ability of T cells. CD19-mCherry overexpression plasmid was constructed, and the virus was packaged in HEK293 (ATCC CRL-1573) cells by a lentiviral packaging system. The medium supernatant was imbibed and then filtered with a 40 μm filter, and the filtered medium supernatant was added to K562 (ATCC CCL 243) tumor cells, which then overexpressed CD19 and mCherry marker proteins through virus infection. PBMC cells were obtained as previously described and cultured in RPMI with 10% FBS 100 U/ml IL2 (novoprotein P60568) after being activated with 1 μg/ml of CD3 and CD28 antibodies. The virus packaged with anti-CD19-41BB (see SEQ ID NO. 1 for the sequence) was infected through the lentivirus packaging system, and the experiment was carried out after amplification. The constructed K562-CD19-mCherry was mixed with K562 cells at a ratio of 1:1, and then mixed with modified anti-CD19-41BB CAR-T cells at different ratios to detect the number of remaining mCherry-positive cells expressed, so as to accurately measure the cytotoxic ability of CAR-T cells against target cells. The specific experimental results are shown in FIG. 2, which shows that, (a) the proportion of surviving K562-CD19 detected by flow cytometry after 8 hours of mixed culture of K562-CD19 and CD19-41BBCAR-T cells at different ratios; (b-d) the levels of granzyme B (GzmB), Interferon-gamma (IFNγ) and Interleukin2 (IL-2) secreted by CD19-41BB CAR-T cells mixed culture with K562-CD19 cells, detected by intracellular staining. It was found that the proportion of mCherry-positive cells in the co-culture system was significantly higher when NAD+ synthesis was inhibited, indicating that the cytotoxic ability of CAR-T cells was attenuated. At the same time, FACS was performed for detecting cytokines as well as proteins GzmB (b), IFNγ (c), and IL-2 (d) secreted during CAR-T cell activation, and it was found that inhibition of NAD+ synthesis significantly reduced the secretion of related cytokines and proteins.

### Embodiment 3

The combination of NAD+-related synthetic precursors and CAR-T therapy enhances the cytotoxic effect of T cells against tumors:

In vivo experiments in mice were performed using CAR-T treatment as a model to verify the feasibility of improving the effectiveness of clinical immunotherapy by supplementing NAD+. In cells with K562-CD19-mCherry, as described previously, luciferase was then overexpressed through the lentiviral system. The constructed K562-CD19-mCherry-luciferase cells were subcutaneously inoculated into immunodeficient mice as target cells to construct solid tumor models, as follows: 5-week-old NSG mice were used for the experiment, and the mice were raised according to the relevant regulations of the Animal Facility of the National Center for Protein Science, and 1*10⁶ K562-CD19-mCherry-luciferase cells were subcutaneously injected to the mice, and four days later, 1*10⁶ modified anti-CD19-41BB CAR-T cells described above were injected into the tail vein of mice or the same amount of saline was injected. After that, NAD+ synthetic precursor nicotinamide (NAM) (Sigma N3376-100G) was used as an NAD+ supplement, and the mice in the NAM experimental group were intraperitoneally injected with 100 μL NAM in saline solution at a concentration of 1 g/ml daily, while the mice in the control group were intraperitoneally injected with 100 μL saline solution daily. Since subcutaneously injected K562 cells also overexpressed Luciferase, intracellular fluorescence can be excited after intraperitoneal injection of the substrate luciferin (PerkinElmer 122799) in mice, and tumor growth in mice can be collected by in vivo imaging. Before CAR-T cells were injected into the tail vein of mice, the fluorescence signal intensity of mouse tumor cells was detected as the starting point, and then the fluorescence intensity of mouse tumor cells was detected every 7 days. For detection, the mice were intraperitoneally injected with 150 μL of D-luciferin potassium salt at a concentration of 10 mg/ml, and 10 minutes later, the fluorescence of tumor cells was detected using IVIS® LuminaIII small animal in vivo imaging system. The stronger the fluorescence, the more tumor cells and the faster the tumor growth. The specific experimental results are shown in 3, which shows that, (a) Mice treated with subcutaneous tumor formation using K562-CD19 cells were treated with saline, Nicotinamide (NAM), CAR-T, or, CAR-T and Nicotinamide (NAM), and in vivo imaging was performed at the time points as shown, n=5; (b) according to the luciferace fluorescence values of mouse tumors, the tumor growth was counted and standardized by the first imaging, n=5; (c) the survival of tumor-bearing mice under different treatments, n=10. It was found that Nicotinamide (NAM) had no significant effect on the fluorescence intensity and tumor growth of tumor cells in immunodeficient mice; the combination of Nicotinamide (NAM) with CAR-T treatment had a significantly better inhibitory effect on tumor cell growth than CAR-T treatment, the fluorescence signal in tumor cells of mice had been undetectable, and the survival time of tumor-bearing mice was also significantly prolonged.

In summary, the present invention effectively overcomes various shortcomings in the field and has a high industrial value.

The above-mentioned embodiments exemplarily illustrate the principles and effects of the present invention, and are not used to limit the present invention. Any skilled person in this field can modify or change the above-mentioned embodiments without departing from the spirit and scope of the present invention. Therefore, all equivalent modifications or changes made by those skilled in the art without departing from the spirit and technical ideas disclosed in the present invention should still be covered by the claims of the present invention.

1. An use of NAD+ and/or an NAD+ agonist and/or an NAD+ inhibitor in the preparation of preparation or kit, wherein the preparation or kit is used for:
(1) regulating the activity of T cells; and/or,
(2) regulating an expression level of CD69 on a surface of T cells; and/or,
(3) regulating a phosphorylation level in T cells; and/or,
(4) treating a disease related to T cell activity.

2. The use according to claim 1, wherein the NAD+ agonist is one or more of NAD+ precursor agonists, a nicotinamide phosphate ribosyltransferase agonist, PARP inhibitors, SIRT inhibitors, a CD38 inhibitors, and NAD+ metabolic enzyme inhibitors;
and/or, the NAD+ inhibitor is one or more of nicotinamide phosphate ribosyltransferase inhibitors, NAD synthase 1 inhibitors, and SIRT agonists.

3. The use according to claim 1, wherein the preparation or kit is used to regulate NAD+ level or NAD+ activity in T cells;
and/or, the regulation includes positive regulation and negative regulation;
and/or, the T cell activity is specifically the cytotoxic ability of T cells, and the cytotoxic ability is a tumor cell-killing ability;
and/or, the T cells also include CAR-T cell and TCR-T cell;
and/or, the disease related to T cell activity is a disease related to insufficient T cell activity or a disease related to excessively high T cell activity;
and/or, the disease related to T cell activity comprises inhibited inflammation, low immune response, tumor, infectious disease, autoimmune disease, T cell-mediated inflammation, and transplant rejection.

4. A regulation method, which is used for:
(1) regulating the activity of T cells; and/or,
(2) regulating an expression level of CD69 on a surface of T cells; and/or,
(3) regulating a phosphorylation level in T cells; and/or,
the regulation method specifically includes: regulating an intracellular level or activity of NAD+.

5. The method according to claim 4, wherein the method specifically comprises: subjecting T cells to a presence of exogenous NAD+, and/or NAD+ inhibitors and/or NAD+ agonists, wherein the NAD+ inhibitors are one or more of nicotinamide phosphate ribosyltransferase inhibitors, NAD synthase 1 inhibitors, and SIRT agonists; the NAD+ agonists are one or more of NAD+, NAD+ precursor agonists, nicotinamide phosphate ribosyltransferase agonists, PARP inhibitors, SIRT inhibitors, CD38 inhibitors, and NAD+ metabolic enzyme inhibitors;
and/or, the regulation is in vitro regulation.

6. The method according to claim 4, wherein the regulation includes positive regulation and negative regulation;
and/or, the T cell activity comprises a cytotoxic ability of T cells, preferably tumor cell killing ability;
and/or, the T cells comprise CAR-T cell and TCR-T cell.

7. A combination preparation, which comprises: T cells, and NAD+ and/or an NAD+ agonist, and/or an NAD+ inhibitor.

8. The combination preparation according to claim 7, wherein the NAD+ inhibitors are one or more of nicotinamide phosphate ribosyltransferase inhibitors, NAD synthase 1 inhibitor, and SIRT agonists;
and/or, the NAD+ agonists are one or more of NAD+, an NAD+ precursor agonist, nicotinamide phosphate ribosyltransferase agonists, PARP inhibitors, SIRT inhibitors, CD38 inhibitors, and NAD+ metabolic enzyme inhibitors;
the T cells comprise CAR-T cells and TCR-T cells.

9. The use of the combination preparation according to claim 7 in the preparation of medicines.

10. The use according to claim 9, wherein the medicines are used to treat diseases related to T cell activity.

11. The use of the combination preparation according to claim 8 in the preparation of medicines.
